(19) United States
(12) Patent Application Publication
(10) Pub. No.: US 2024/0335403 A1
(43) Pub. Date: Oct. 10, 2024

(54) COMPOSITIONS AND METHODS FOR METABOLIC HEALTH

(71) Applicant: Glucose Health, Inc., Bentonville, AR (US)

(72) Inventor: Murray Stewart Fleming, Victoria (CA)

(21) Appl. No.: 18/742,991

(22) Filed: Jun. 13, 2024

Related U.S. Application Data
(63) Continuation of application No. PCT/US2022/082178, filed on Dec. 21, 2022.
(60) Provisional application No. 63/293,356, filed on Dec. 23, 2021.

(51) Int. Cl. A61K 31/192; A61K 31/555; A61K 31/718; A61P 3/06; A61P 3/10

(52) U.S. Cl. CPC A61K 31/192; A61K 31/555; A61K 31/718; A61P 3/06; A61P 3/10

(57) ABSTRACT

Compositions for managing post-prandial metabolic changes, including glycemic and lipid responses, and related methods of manufacture and use. A composition for attenuating post-prandial glycemic response (GR) and lipid response (LR) reduces risk of chronic metabolic diseases. Certain ingredients of the composition include resistant dextrin, corosolic acid, and chromium(III) picolinate, which may be combined together using a wet agglomeration or a dry mixing step. The agglomerated or dryly mixed ingredients may then be intimately dry blended with a flavored matrix to improve palatability. The composition may be consumed before, during, and/or after a meal for attenuation of post-prandial rise in plasma glucose, triglyceride levels, and cholesterol levels and reduction of risk of chronic metabolic diseases.

### BACKGROUND

### Field

The disclosure relates to compositions and methods for maintaining or improving health of an individual by attenuating post-prandial glycemic and lipid increases and/or lowering levels of plasma glucose and lipids. Compositions include resistant dextrin, corosolic acid, and chromium(III) picolinate in amounts effective for reducing risk of developing and worsening metabolic diseases.

### Description of the Related Art

Plasma glucose, insulin, and lipid levels increase after meals, and increases and variabilities in these factors have been implicated in the development of chronic metabolic diseases such as obesity, type 2 diabetes mellitus (T2DM), and cardiovascular disease (CVD) (Blaak et al. Obes Rev. 2012 October; 13 (10): 923-84). Obesity is a result of excess weight gain, which is caused by consuming more calories than the body requires for its basal metabolic functions and additional activities in which an individual is involved. Losing weight is difficult for many individuals, as reduced caloric intake often induces cravings for high glycemic index foods that reduce adherence to weight loss regimens. These cravings, particularly when satiated by high glycemic index foods, cause significant post-prandial increases in plasma glucose, insulin, and lipid levels, which further increases the probability of these individuals developing metabolic diseases.

Certain compounds and compositions have been researched for evaluation of their ability to combat the development and progression of metabolic diseases. For example, soluble dietary fibers help control post prandial blood glucose, lower serum lipids, regulate the digestive system, increase micronutrient absorption, and may have a role in the prevention of cardiovascular disease (Slavin J L et al. J Int Med Res. January-February 2009; 37(1): 1-17). Banaba leaf (e.g., Lagerstroemia speciosa L.) has been used in folk medicine to treat diabetes, and the hypoglycemic effects of Banaba leaf has been attributed to both corosolic acid as well as ellagitannins. The beneficial effects of corosolic acid with respect to various aspects of glucose and lipid metabolism appear to involve multiple mechanisms, including enhanced cellular uptake of glucose, impaired hydrolysis of sucrose and starches, decreased gluconeogenesis and the regulation of lipid metabolism. (Stohs S J et al. Phytother Res. 2012 March; 26 (3): 317-24). The trace element chromium has been the subject of research, which describes the use of a chromium salt, either alone or in combination with other ingredients, in lowering blood levels of lipids or controlling blood glucose levels.

Despite these potential health benefits of soluble dietary fiber, corosolic acid, and chromium individually, many individuals, including obese individuals and individuals at risk of developing metabolic diseases, may not consume these ingredients in amounts effective to stave off metabolic disease. Further, efforts to introduce these ingredients into an individual's diet may fail as the individual struggles to change meal preparation, dietary, exercise, and lifestyle habits. In addition, the individual's palate and taste preferences may preclude the adoption of these ingredients as part of a dietary change.

While knowledge of the benefits of these ingredients individually exists, it is not known whether a combination of these three ingredients would provide health benefits that exceed the benefits of the individual components. It is also not known what particular amounts of these three ingredients together would be effective for a dose and a treatment regimen. Further, even if particular formulations of these three ingredients together were found to be beneficial, they may not be acceptably palatable to humans and efforts to make these ingredients palatable may be thwarted by the potential introduction of undesired or unhealthy ingredients. As such, the health benefits of combinations of these three ingredients are unknown, and further, attempts to make such combinations palatable to humans by introducing flavor agents would not necessarily result in a combination that is clinically effective and acceptably palatable.

Accordingly, there is a need for improved compositions and methods for managing post-prandial levels of plasma glucose and lipids. The present disclosure addresses this unmet need.

### SUMMARY

In one aspect, the disclosure provides compositions for managing metabolic health, the compositions including resistant dextrin, an extract of Banaba leaf (Lagerstroemia speciosa L.) with standardized corosolic acid, chromium(III) picolinate, and a flavored matrix to improve palatability of the composition. Upon an administration of a dose of a composition to an individual prior to, or concurrently with, consuming foods containing carbohydrates and/or dietary fats, the composition attenuates a post-prandial increase of, and/or lowers plasma concentrations of, glucose, triglycerides, and cholesterol in the individual. The dose of the composition may be formulated as a food or beverage for eating or drinking, for example, as a dietary supplement and/or a functional food. The composition is palatable and is formulated and dosed to be effective at managing glucose and lipid levels and preventing onset and progression of metabolic diseases.

In another aspect, the disclosure provides methods for managing metabolic health, the methods including administering a dose of a composition to an individual. The composition administered includes resistant dextrin, an extract that includes corosolic acid, and chromium(III) picolinate. Upon administering the dose of the composition to the individual prior to, or concurrently with, consuming foods containing carbohydrates and/or dietary fats, a post-prandial increase of plasma concentrations of glucose, triglycerides, and cholesterol is attenuated, and/or plasma concentrations of glucose, triglycerides, and cholesterol are lowered, in the individual.

In yet another aspect, the disclosure provides methods for manufacturing compositions for metabolic health management, the methods including wetly agglomerating or dry mixing resistant dextrin, an extract of standardized corosolic acid, and chromium(III) picolinate to form an agglomerate or a dry mixture. The methods may further include dry blending the agglomerate or the dry mixture with a flavored matrix to improve palatability of the composition. The flavored matrix may include a flavor agent, a flavor enhancing agent, or both.

In particular implementations, the composition may include resistant dextrin, in the form of soluble corn fiber, a type of dietary fiber; an extract of Lagerstroemia speciosa L. standardized to about 1% corosolic acid, and the trace element chromium in the form of chromium(III) picolinate.

In particular implementations, certain amounts and/or ranges of ingredients of the composition may be used for an optimal or beneficial formulation. For example, in certain implementations, one dose of the composition may include about 4500 mg to about 5000 mg of the resistant dextrin, about 0.48 mg to about 1.00 mg of the Lagerstroemia speciosa L. extract, and about 35 μg to about 200 μg of the chromium(III) picolinate. In some implementations, one dose of the composition may include more specific or optimal amounts within these ranges; for example, the dose of the composition may include about 4500 mg of the resistant dextrin, about 0.48 mg of the Lagerstroemia speciosa L. extract, and about 35 μg of the chromium(III) picolinate. Certain ranges for the ingredients may be more optimal or beneficial than others and may provide health benefits that exceed the benefits of the ingredients individually.

In particular implementations, certain amounts and/or ranges of ingredients of the composition may be used for an optimal or beneficial formulation. For example, in certain implementations, one dose of the composition may include about 4500 mg to about 5500 mg of the resistant dextrin, about 0.48 mg to about 1.00 mg of the Lagerstroemia speciosa L. extract, and about 35 μg to about 200 μg of the chromium(III) picolinate. In some implementations, one dose of the composition may include more specific or optimal amounts within these ranges; for example, the dose of the composition may include about 5000 mg of the resistant dextrin, about 0.48 mg of the Lagerstroemia speciosa L. extract, and about 35 μg of the chromium(III) picolinate. Certain ranges for the ingredients may be more optimal or beneficial than others and may provide health benefits that exceed the benefits of the ingredients individually.

Accordingly, in yet another aspect, the disclosure provides compositions for metabolic health management, manufactured by a method, the method including wetly agglomerating or dry mixing a plurality of ingredients to form an agglomerate or a dry mixture, and further, dry blending the agglomerate or the dry mixture with a flavored matrix to improve palatability of the compositions. The plurality of ingredients includes resistant dextrin, a Lagerstroemia speciosa L. extract standardized to about 1% corosolic acid, and chromium(III) picolinate. The method further includes dividing the agglomerate or dry mixture and flavored matrix into a plurality of doses, and each dose of the plurality of doses includes about 4500 mg to about 5000 mg of the resistant dextrin, about 0.48 mg to about 1.00 mg of the Lagerstroemia speciosa L. extract, and about 35 μg to about 200 μg of the chromium(III) picolinate.

Accordingly, in yet another aspect, the disclosure provides compositions for metabolic health management, manufactured by a method, the method including wetly agglomerating or dry mixing a plurality of ingredients to form an agglomerate or a dry mixture, and further, dry blending the agglomerate or the dry mixture with a flavored matrix to improve palatability of the compositions. The plurality of ingredients includes resistant dextrin, a Lagerstroemia speciosa L. extract standardized to about 1% corosolic acid, and chromium(III) picolinate. The method further includes dividing the agglomerate or dry mixture and flavored matrix into a plurality of doses, and each dose of the plurality of doses includes about 4500 mg to about 5500 mg of the resistant dextrin, about 0.48 mg to about 1.00 mg of the Lagerstroemia speciosa L. extract, and about 35 μg to about 200 μg of the chromium(III) picolinate. In some embodiments, the method includes dividing the agglomerate or dry mixture and flavored matrix into a plurality of doses, and each dose of the plurality of doses includes about 5000 mg of the resistant dextrin, about 0.48 mg of the Lagerstroemia speciosa L. extract, and about 35 μg of the chromium(III) picolinate.

Another object of the present disclosure is to provide optimized and improved compositions and methods that may be used as part of a dietary supplement or functional food or beverage for metabolic health management, and other benefits.

Other objects, features and advantages of the present disclosure will become apparent from the following detailed description taken in conjunction with the accompanying drawings.

### BRIEF DESCRIPTION OF THE DRAWINGS

Although the characteristic features of the disclosure will be particularly pointed out in the claims, exemplary implementations of the disclosure and manners in which they may be made and used may be better understood after a review of the following description, taken in connection with the accompanying drawings, wherein like numeral annotations are provided throughout.

FIG. 1 depicts a flowchart of a metabolic response to a meal which may lead to increased risk of metabolic disease.

FIG. 2A depicts a flowchart of an attenuated metabolic response to a meal as a result of administering a dose of a composition of the present disclosure.

FIG. 2B depicts a flowchart of a decrease of plasma glucose, triglycerides, and cholesterol as a result of administering a dose of a composition of the present disclosure.

FIG. 3A depicts a flowchart of a control process for measuring a metabolic response to a meal.

FIG. 3B depicts a flowchart of a test process for measuring an attenuation of a metabolic response to a meal as a result of administering a dose of a composition of the present disclosure, and adjusting the dose based on an evaluation of the attenuation.

FIG. 4A depicts a flowchart of a process for manufacturing a composition of the present disclosure.

FIG. 4B depicts a flowchart of a process for manufacturing a composition, including a palatable composition, of the present disclosure.

### DETAILED DESCRIPTION

Reference is made herein to the attached drawings. Like reference numerals may be used in the drawings to indicate like or similar elements of the description. The figures are intended for representative purposes, are not drawn to scale, and should not be considered limiting.

Unless otherwise defined herein, terms and phrases used in connection with the present disclosure shall have the meanings that are commonly understood by those of ordinary skill in the art.

As used in the description and in the claims, the terms “comprising” and “comprises” do not exclude other elements or steps. Where an indefinite or definite article is used when referring to a singular noun, e.g., “a,” “an,” or “the,” this includes a plural of that noun unless something else is specifically stated. Furthermore, the terms first, second, third, and the like in the description and in the claims, are used for distinguishing between elements and not necessarily for describing a sequential or chronological order. It is to be understood that the terms so used are interchangeable under appropriate circumstances and that the implementations of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations that are directed to that value or parameter per se. The term “about” refers to values within 1, 2, 3, 4, 5, 6, 7, 8, 9, 10, 11, 12, 13, 14, 15, 16, 17, 18, 19, or 20 percent of that value.

As used herein, the term “acid,” when used to describe a compound or ingredient of a composition (e.g., corosolic acid, malic acid, citric acid, fumaric acid, tartaric acid, etc.), includes both protonated and non-protonated forms of the acid. Unless described or claimed otherwise, or if such implementations would be unattainable and/or undesirable, use of the term “acid” to describe or claim a compound also includes salts of the compound that are complexed and/or ionically bonded with a counter-ion, such as sodium, potassium, lithium, calcium, magnesium, zinc, and/or iron.

As used herein, the term “extract,” when used to describe a substance or ingredient of a composition (e.g., a Lagerstroemia speciosa L. extract standardized to about 1% corosolic acid), includes any extract present as an isolated form as well as any extract present as a pharmaceutically acceptable and/or food acceptable extract and/or salt. Exemplary salts may be a compound of the extract complexed and/or ionically bonded with a counter-ion, such as sodium, potassium, lithium, calcium, magnesium, zinc, and/or iron. The extract may include a compound within a suitable carrier, solvent, diluent, and/or matrix, used to extract the compound from a starting material and/or to hold, solubilize, and/or dilute the compound prior to and/or subsequent to formulation of a composition that includes the compound and/or the extract.

As used herein, the term “flavor agent” refers to any compound and/or mixture of compounds that contributes to a quality or characteristic of a flavor of a composition (e.g., a fruit flavor, a banana flavor, an apple flavor, etc.). The flavor agent may be derived from or include essences, extracts, oils, and the like, and may be derived from plant sources.

As used herein, the term “flavor enhancing agent” refers to any compound and/or mixture of compounds that contributes to a quantity of a flavor of a composition, and when present in a composition, may increase and/or enhance an individual's perception of the flavor (e.g., citric acid, malic acid, sucralose, etc.).

As used herein, the term “flavored matrix” refers to one or more matrices that includes a flavor agent, multiple flavor agents, a flavor enhancing agent, multiple flavor enhancing agents, or any combination thereof.

As used herein, the term “resistant dextrin” refers to short chain glucose polymers which are strongly resistant to human digestive enzymes. While the resistant dextrin may be derived through a process of hydrolyzation from any of a plurality of sources of starch (e.g., potatoes, wheat, rice, tapioca, etc.), in particular implementations, it may be derived from corn starch and be a soluble corn fiber, a form of dietary fiber.

As used herein, the term “Lagerstroemia speciosa L.” refers to a tropical flowering tree in the Lythraceae family, and may be commonly known as giant crepe myrtle, Queen's crepe myrtle, Pride of India, or Banaba leaf.

As used herein, the term “chromium(III) picolinate” (synonyms: Chromium picolinate 14639-25-9, Chromium (III) Pyridine-2-carboxylate, Chromium (III) Picolinate, Chromium Picolinate, and CrPic3; molecular formula: C₁₈H₁₂CrN₃O₆) refers to a coordination complex comprised of trivalent chromium (i.e., chromium(III)) and picolinic acid.

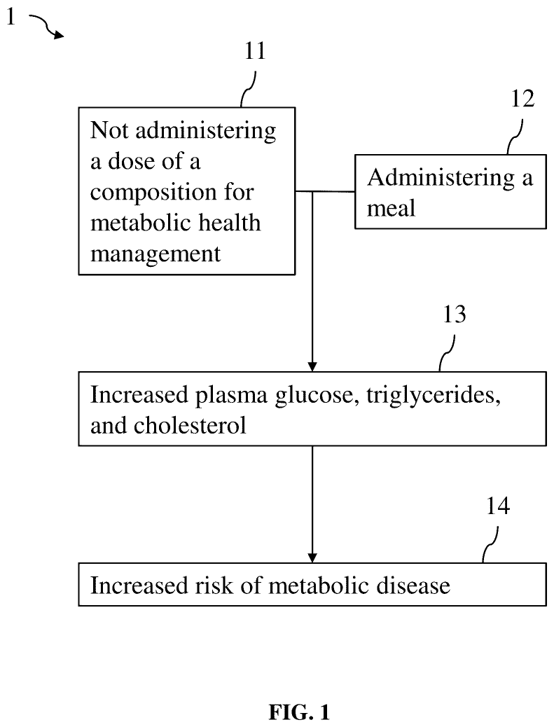

Referring now to FIG. 1, there is depicted a flowchart of a metabolic response to a meal which may lead to increased risk of metabolic disease. A problem may arise when an obese or other individual at risk of developing metabolic disease undergoes a process 1 whereby they are administered a meal 12 without administering a dose of a composition of the present disclosure. As a result, the individual experiences 13 increased plasma glucose, triglycerides, and cholesterol, and may be at a greater risk of developing metabolic disease 14. Since this individual does not receive 11 the dose of the composition before, during, or after the meal, the individual may develop metabolic disease and/or T2DM. At least these individuals would benefit from consuming compositions of the present disclosure.

### Compositions

In various implementations, a composition for managing metabolic health includes resistant dextrin, a Lagerstroemia speciosa L. extract standardized to about 1% corosolic acid, chromium(III) picolinate, and a flavored matrix to improve palatability of the composition. Upon an administration of a dose of the composition to an individual prior to, or concurrently with, foods containing carbohydrates and/or dietary fats, the composition attenuates a post-prandial increase of, and/or lowers plasma concentrations of, glucose, triglycerides, and cholesterol in the individual. The dose of the composition may be formulated as a food or beverage for eating or drinking, for example, as a dietary supplement or functional food or beverage. The composition is palatable and effective at managing glucose and lipid levels and preventing onset and progression of metabolic diseases. In some embodiments, the dose of the composition of the instant disclosure may attenuate post-prandial increase of, and/or lower plasma concentrations of, glucose, triglycerides, and cholesterol in the individual by about 5%, 10%, 15%, 20%, 25%, 30%, 35%, 40%, 45%, 50%, 55%, 60%, 65%, 70%, 75%, 80%, 85%, 90%, 95%, or 100%, or within a range defined by any two of the aforementioned percentages. For example, the composition of the instant disclosure may attenuate post-prandial increase of, and/or lower plasma concentrations of, glucose, triglycerides, and cholesterol in the individual by from about 5% to about 90%, from about 10% to about 80%, from about 20% to about 70%, from about 30% to about 60%, or from about 40% to about 50%.

In certain implementations, the composition may include the resistant dextrin, in the form of soluble corn fiber, the Lagerstroemia speciosa L. extract standardized to about 1% corosolic acid, the chromium(III) picolinate, and the flavored matrix, and may be completely, essentially, or substantially devoid of any other compounds, ingredients, and/or additives. In such implementations, the composition may be simple and may retain the health benefits of other implementations of the composition. In this respect, the disclosure provides a relatively simple yet effective composition that is capable of benefitting individuals at risk of developing or worsening metabolic diseases without necessarily including additional compounds, ingredients, and/or additives.

In various implementations, resistant dextrin is included in the compositions and may be present at a higher concentration per dose compared to previous formulations. The resistant dextrin can provide acute and long-term metabolic improvements in a variety of individuals, including individuals having or at risk of developing T2DM and its related complications such as cardio-vascular diseases, neuropathy (nerve damage), nephropathy (kidney damage), retinopathy (eye damage) and other complications. Particular metrics that may be representative of such improvements may include reductions in hemoglobin A1c, fasting and post-prandial glycemia, insulinemia, cholesterol, and triglycerides. In addition, resistant dextrin may be helpful in weight control and avoiding obesity by promoting a feeling of fullness which may dissuade individuals from departing from a diet or lifestyle plan.

In various implementations, the resistant dextrin may be present in the composition at an amount between about 4500 mg per dose and about 5000 mg per dose. In some implementations, the resistant dextrin may be present at an amount between about 4500 mg per dose and about 4900 mg per dose, between about 4500 mg per dose and about 4800 mg per dose, between about 4500 mg per dose and about 4700 mg per dose, between about 4500 mg per dose and about 4600 mg per dose, or may be present in the composition at an amount of about 4500 mg per dose. In some implementations, the resistant dextrin may be present in the composition at an amount of 4500 mg per dose.

In various implementations, the resistant dextrin may be present in the composition at an amount between about 3500 mg per dose and about 6500 mg per dose. In some implementations, the resistant dextrin may be present at an amount between about 4500 mg per dose and about 5500 mg per dose, between about 4900 mg per dose and about 5100 mg per dose, between about 4950 mg per dose and about 5050 mg per dose, between about 4980 mg per dose and about 5020 mg per dose, or may be present in the composition at an amount of about 5000 mg per dose. In some implementations, the resistant dextrin may be present in the composition at an amount of 5000 mg per dose. In some embodiments, the resistant dextrin in the composition at an optimal amount of 5000 mg per dose. In some embodiments, the resistant dextrin in the composition at an optimal amount of 5000 mg per dose when in combination with a chromium(III) compound and/or corosolic acid.

An extract of the plant Laegerstroemia speciosa L. standardized to 1% corosolic acid is included in the compositions and may be present at an optimum or beneficial concentration per dose compared to previous formulations. The corosolic acid provides acute and long-term metabolic improvements in a variety of individuals, including individuals having or at risk of developing T2DM. Corosolic acid may impact numerous biological processes, and may provide antidiabetic, anti-inflammatory, antiproliferative, and/or protein kinase C inhibition activity. The beneficial effects with respect to various aspects of glucose and lipid metabolism appear to involve multiple mechanisms, including enhanced cellular uptake of glucose, impaired hydrolysis of sucrose and starches, decreased gluconeogenesis, and the regulation of lipid metabolism. These effects may be mediated by PPAR and other signal transduction factors. Corosolic acid, and other constituents may be beneficial in addressing the symptoms associated with metabolic syndrome, as well as offering other health benefits (Miura et al. Evid Based Complement Alternat Med. 2012; 2012:871495.). Increased consumption of corosolic acid may help manage diabetes and other metabolic diseases.

Corosolic acid of the composition may be present in an extract of the plant Lagerstroemia speciosa L. standardized to about 1% corosolic acid and/or exactly 1% corosolic acid. Lagerstroemia speciosa L. (may also be known as giant crepe-myrtle, Queen's crepe-myrtle, Pride of India, or Banaba leaf) is a species of Lagerstroemia native to tropical southern Asia.

In various implementations, the Lagerstroemia speciosa L. extract (standardized to about 1% corosolic acid and/or 1% corosolic acid) are administered at a therapeutically effective dosage. Generally, a daily dose may be from about 0.25 μg/kg to about 120 μg/kg or more of body weight, from about 0.5 μg/kg or less to about 70 μg/kg, from about 1.0 μg/kg to about 50 μg/kg of body weight, or from about 1.5 μg/kg to about 10 μg/kg of body weight. Thus, for administration to a 70 kg person, the dosage range would be from about 17 μg per day to about 8000 μg per day, from about 35 μg per day or less to about 7000 μg per day or more, from about 70 mg per day to about 6000 μg per day, from about 100 μg per day to about 5000 μg per day, or from about 200 μg to about 3000 μg per day. The amount of active compound administered will, of course, be dependent on the subject and disease state being treated, the severity of the affliction, and//or the manner and schedule of administration.

In various implementations, the Lagerstroemia speciosa L. extract (standardized to about 1% corosolic acid and/or 1% corosolic acid) may be present in the composition at an amount between about 0.48 mg per dose and about 1.00 mg per dose. In implementations, the Lagerstroemia speciosa L. extract may be present at an amount between about 0.48 mg per dose and about 0.90 mg per dose, between about 0.48 mg per dose and about 0.80 mg per dose, between about 0.48 mg per dose and about 0.70 mg per dose, between about 0.48 mg per dose and about 0.60 mg per dose, or may be present in the composition at an amount of about 0.48 mg per dose. In implementations, the Lagerstroemia speciosa L. extract may be present in the composition at an amount of 0.48 mg per dose.

In implementations, a Lagerstroemia speciosa L. extract standardized to a different concentration of corosolic acid (i.e., other than about 1%), such as about 2%, may be used to produce and/or included in a composition of the disclosure, and the volume of the different concentration adjusted to product the same amount of corosolic acid per dose. It should be understood that the amount of the corosolic acid in the composition having the 2% corosolic acid extract may remain substantially or completely unchanged compared to the composition with having the 1% corosolic acid extract. As a non-limiting example, if 0.48 mg of an extract containing 1% corosolic acid is included in a dose, and if an extract containing 2% corosolic acid is included in the dose instead, then 0.24 mg of the 2% corosolic acid extract should be used to produce the same or about the same amount of corosolic acid in the dose, and amounts of other elements of the dose (e.g., the resistant dextrin) may be adjusted to accommodate the change in the amount of the corosolic acid extract as needed. The same principle applies regardless of whether a 1% corosolic acid extract, a 2% corosolic acid extract, a 10% corosolic acid extract, or another concentration of corosolic acid in an extract is used to product the composition.

Chromium (III) picolinate, a source of bioavailable chromium(III), is included in the compositions and may be present at an optimum or beneficial concentration per dose compared to previous formulations. The chromium(III) picolinate provides acute and long-term metabolic improvements in a variety of individuals, including individuals having or at risk of developing T2DM. Chromium (III) may play an essential role in regulating carbohydrate metabolism and may help regulate glycemic control. Increased consumption of chromium(III) picolinate may help manage diabetes and other metabolic diseases.

In various implementations, the chromium(III) compound (e.g., chromium(III) picolinate) may be present in the composition at an amount between about 35 μg and about 200 μg per dose. In implementations, the chromium(III) compound may be present at an amount between about 35 μg and about 175 μg per dose, between about 35 μg and about 150 μg per dose, between about 35 μg and about 125 μg per dose, between about 35 μg and about 100 μg per dose, or may be present in the composition at an amount of about 35 μg per dose. In implementations, the chromium(III) compound may be present in the composition at an amount of 35 μg per dose.

In some embodiments, the resistant dextrin may be present in the composition at an amount between about 3500 mg per dose and about 6500 mg per dose, the Lagerstroemia speciosa L. extract (standardized to about 1% corosolic acid and/or 1% corosolic acid) may be present in the composition at an amount between about 0.2 mg per dose and about 2.0 mg per dose, and the chromium(III) compound may be present at an amount between about 15 μg and about 250 μg per dose. In some embodiments, the resistant dextrin may be present in the composition at an amount between about 4500 mg per dose and about 5500 mg per dose, the Lagerstroemia speciosa L. extract (standardized to about 1% corosolic acid and/or 1% corosolic acid) may be present in the composition at an amount between about 0.48 mg per dose and about 4.0 mg per dose, and the chromium(III) compound may be present at an amount between about 35 μg and about 200 μg per dose. In some embodiments, the resistant dextrin may be present in the composition at an amount of about 500 mg per dose, the Lagerstroemia speciosa L. extract (standardized to about 1% corosolic acid and/or 1% corosolic acid) may be present in the composition at an amount of about 0.48 mg per dose, and the chromium(III) compound may be present at an amount of about 35 μg per dose.

### Additives

In various implementations, one or more additives may be included in a composition of the disclosure to improve the effectiveness, palatability, shelf life, and/or other properties or characteristics of the composition or a nutritional product comprising the composition. Such additives may include a sweetener, such as a non-nutritive sweetener, or another compound or substance for improving the composition or nutritional product.

In various implementations, highly bioavailable vitamins and/or minerals may be included in the composition for maintaining and/or improving the individual's health. For example, manganese citrate (a source of manganese), zinc picolinate (a source of zinc), vitamin B1 (thiamine), vitamin B6 (pyridoxine), vitamin B7 (biotin), vitamin B12 (cyanocobalamin), or any combination thereof may be included in a composition and/or a dose of the present disclosure. Particular amounts of these and/or other additives may be adjusted or optimized for maximum effectiveness.

Manganese (Mn) is an essential element that is involved in the synthesis and activation of many enzymes and in the regulation of the metabolism of glucose and lipids in humans. In addition, Mn is one of the required components for Mn superoxide dismutase (MnSOD) that is mainly responsible for scavenging reactive oxygen species (ROS) in mitochondrial oxidative stress. Studies have found that ROS generation, oxidative stress, and inflammation are critical for the pathogenesis of metabolic diseases. In addition, deficiency in dietary Mn as well as excessive Mn exposure could increase ROS generation and result in further oxidative stress. Accordingly, in various implementations of compositions disclosed herein, manganese (e.g., manganese citrate) may be present in amounts sufficient to prevent development or progression of metabolic diseases.

Zinc (Zn) is an essential mineral in a number of physiological processes, and impacts blood glucose, triglyceride levels, and cholesterol levels. In addition, zinc supplementation may improve glycemic control and other cardiometabolic and anthropometric parameters and help with disease progression in prediabetic individuals. Accordingly, in various implementations of compositions disclosed herein, zinc (e.g., zinc picolinate) may be present in amounts sufficient to prevent development or progression of metabolic diseases.

Vitamin B1 (thiamine) acts as a coenzyme for transketolase (Tk) and for the pyruvate dehydrogenase and a-ketoglutarate dehydrogenase complexes, enzymes which play a fundamental role for intracellular glucose metabolism. The relationship between thiamine and diabetes mellitus (DM) has been reported in the literature, and thiamine levels and thiamine-dependent enzyme activities have been reduced in DM. Thiamine and its derivatives prevent the activation of certain biochemical pathways (increased flux through the polyol pathway, formation of advanced glycation end-products, activation of protein kinase C, and increased flux through the hexosamine biosynthesis pathway) induced by hyperglycemia in DM. Thiamine also has a role in the diabetic endothelial vascular diseases (micro and macroangiopathy), lipid profile, retinopathy, nephropathy, cardiopathy, and neuropathy. Accordingly, in various implementations of compositions disclosed herein, vitamin B1 (thiamine) may be present in amounts sufficient to prevent development or progression of metabolic diseases.

Vitamin B6 in coenzyme forms performs a wide variety of functions in the body and is extremely versatile, with involvement in more than 100 enzyme reactions, mostly concerned with protein metabolism. Both pyridoxal 5′ phosphate (PLP) and pyridoxamine 5′ phosphate (PMP) are involved in amino acid metabolism, and PLP is also involved in the metabolism of one-carbon units, carbohydrates, and lipids. In addition, vitamin B6 also plays a role in cognitive development through the biosynthesis of neurotransmitters and in maintaining normal levels of homocysteine, an amino acid in the blood. Vitamin B6 is also involved in gluconcogenesis and glycogenolysis, immune function (for example, it promotes lymphocyte and interleukin-2 production), and hemoglobin formation. Accordingly, in various implementations of compositions disclosed herein, vitamin B6 (pyridoxine) may be present in amounts sufficient to prevent development or progression of metabolic diseases.

It is known that chromium and vitamin B7 (biotin) play essential roles in regulating carbohydrate metabolism, and compositions that include chromium picolinate and biotin, administered as an adjuvant to current prescription anti-diabetic medication, can improve glycemic control in overweight to obese individuals with type 2 diabetes, especially those patients with poor glycemic control on oral therapy. Accordingly, in various implementations of compositions disclosed herein, biotin may be present in amounts sufficient to prevent development or progression of metabolic diseases.

Vitamin B12 is an essential micronutrient required for optimal hemopoietic, neuro-cognitive, and cardiovascular function. Biochemical and clinical vitamin B12 deficiency has been demonstrated to be highly prevalent among patients with type 1 and type 2 diabetes mellitus. Vitamin B12 deficiency presents with diverse clinical manifestations ranging from impaired memory, dementia, delirium, peripheral neuropathy, sub-acute combined degeneration of the spinal cord, megaloblastic anemia, and pancytopenia. Accordingly, in various implementations of compositions disclosed herein, vitamin B12 (cyanocobalamin) may be present in amounts sufficient to prevent development or progression of metabolic diseases.

In various implementations, caffeine may be omitted from a composition, a meal, a beverage, and/or a treatment of the present disclosure to improve or maintain the effectiveness of the dose consumed. In certain instances, for example, if an individual is diagnosed with T2DM or is pre-diabetic, post-prandial levels of plasma glucose, triglycerides, and/or cholesterol of the individual may be exaggerated or more severely increased if caffeine is consumed with a meal. Similarly, it may be recommended that the individual abstain from caffeine consumption before, during, and/or after consuming a composition of the present disclosure. In this manner, the effects of certain ingredients of the composition are not countered or interfered with by the presence of caffeine, whether in the composition or in another substance such as another dietary supplement, coffee, tea, soda, energy drinks, and the like.

In implementations, vitamin B7 (biotin) may be included as an additive in the composition to enhance or contribute to the management of carbohydrate metabolism. For example, biotin may be included along with a source of bioavailable chromium(III), such as chromium(III) picolinate, and the combination may be administered with one or more doses of the composition, e.g., as an adjuvant to anti-diabetic medication, to improve glycemic control in overweight and obese individuals with T2DM. Clinical readouts such as glycosylated hemoglobin (HbA1c), fasting glucose, and/or lipid levels, may be used to monitor an individual's response to the combination and the amount of biotin in the composition or dose may be adjusted or optimized.

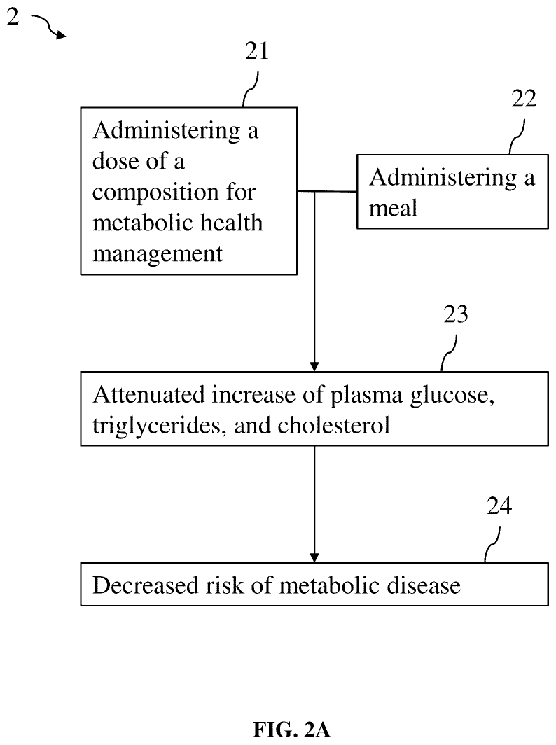

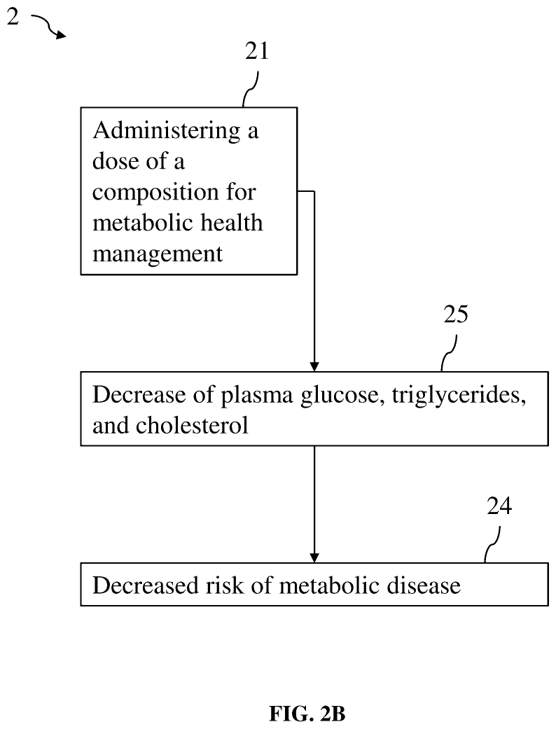

Referring now to FIGS. 2A and 2B, there are depicted flowcharts of an attenuated metabolic response to a meal (FIG. 2A) and a decrease of plasma glucose, triglycerides, and cholesterol (FIG. 2B) as a result of administering a dose of a composition of the present disclosure. A method 2 for attenuating an increase of plasma glucose, triglycerides, and cholesterol includes administering 21 a dose of a composition of the present disclosure to an individual before, during, and/or after administering 22 a meal to the individual. After the meal, the individual experiences an attenuated increase 23 of plasma glucose, triglycerides, and cholesterol (FIG. 2A). Regardless of whether the meal is administered, and including instances wherein the meal is not administered, a decrease 25 of plasma glucose, triglycerides, and cholesterol may be achieved as a result of administering 21 the dose of the composition to the individual (FIG. 2B). Regardless of whether the meal is administered (FIG. 2A) or not (FIG. 2B), the individual has a decreased risk 24 of metabolic disease.

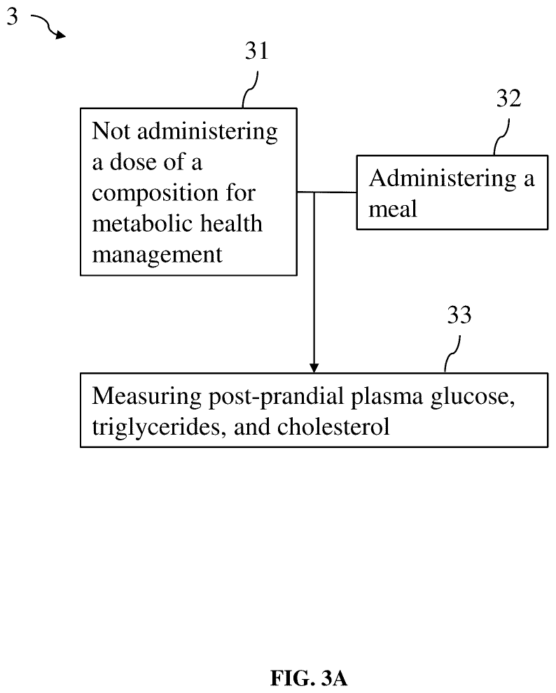

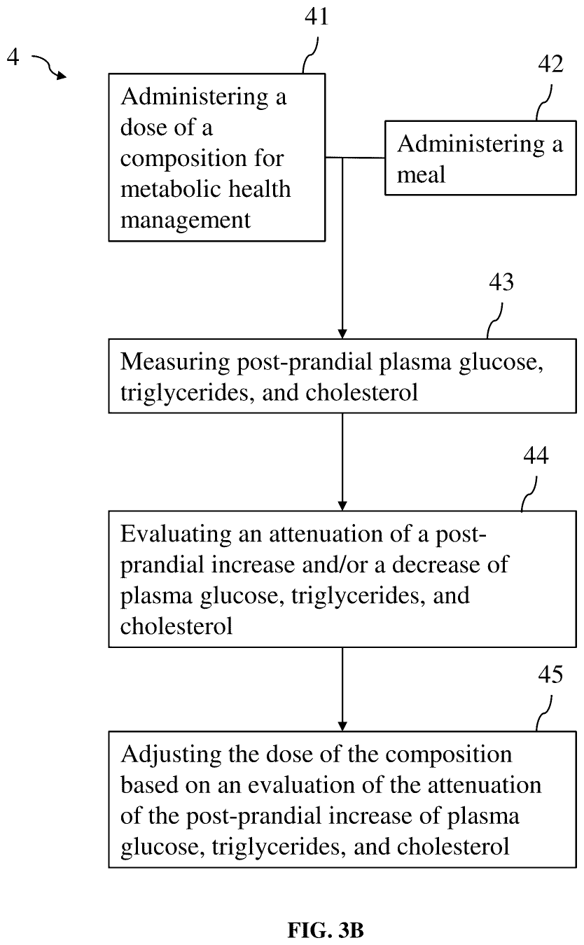

Referring now to FIGS. 3A and 3B, there are depicted a flowchart of a control process for measuring a metabolic response to a meal (FIG. 3A) and a flowchart of a test process for measuring an attenuation of a metabolic response to a meal as a result of administering a dose of a composition of the present disclosure, and adjusting the dose based on an evaluation of the attenuation (FIG. 3B). In implementations, a control process 3 and a test process 4 may be helpful for evaluating an individual's response to a dose of a composition of the disclosure combined with a meal, and adjusting or optimizing the dose based on the response. The control process 3 includes not administering 31 a dose of a composition to an individual, administering a meal 32 to the individual, and measuring 33 levels of plasma glucose, triglycerides, and cholesterol in the individual after the meal. The test process 4 includes administering 41 a dose of a composition, administering a meal 42, and measuring 43 levels of plasma glucose, triglycerides, and cholesterol in the individual after the meal. The attenuation of the post-prandial increase of plasma glucose, triglycerides, and cholesterol, and/or a decrease of plasma glucose, triglycerides, and cholesterol, may be evaluated 44 and the dose of the composition adjusted 45 based on the evaluation. The measurements, evaluations, and dose adjustments may be performed using established biomedical systems, methods, and informed decision-making processes.

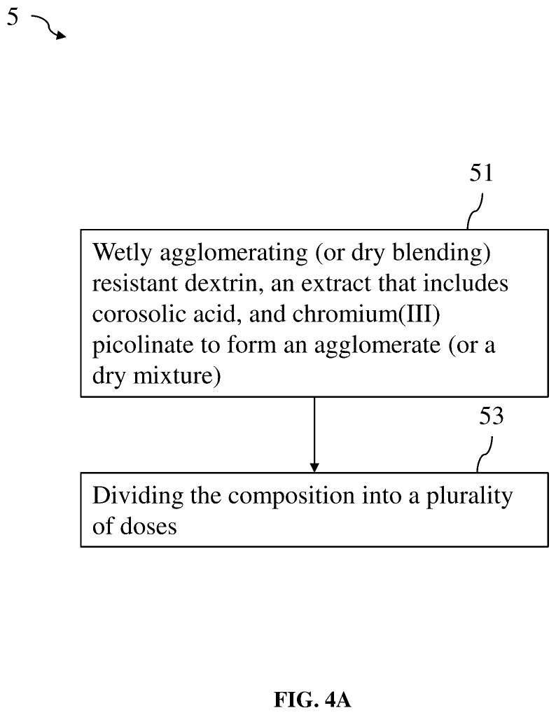

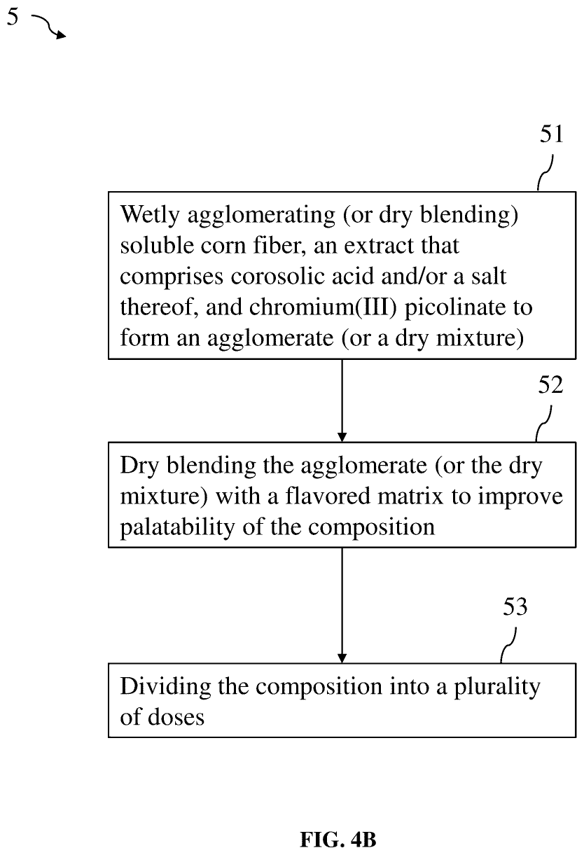

Referring now to FIGS. 4A and 4B, there are depicted a flowchart of a process for manufacturing a composition (FIG. 4A) and a flowchart of a process for manufacturing a composition, including a palatable composition (FIG. 4B) of the present disclosure. In various implementations, a composition of the present disclosure may be manufactured using an agglomeration process, such as a wet agglomeration process. A method 5 for manufacturing a composition may include wetly agglomerating (or dry blending) 51 resistant dextrin, an extract that includes corosolic acid, and chromium(III) picolinate to form an agglomerate (or a dry mixture). The method 5 may involve dividing the composition 53 into a plurality of doses to facilitate packaging, delivery, and use of the composition by consumers, patients, doctors, nutritionists, and the like.

As part of the agglomeration step 51, the resistant dextrin, the extract that includes corosolic acid, the chromium(III) picolinate, and other components may be wetly agglomerated to a maximum mass density per cubic centimeter in one processing step. In certain implementations, the maximum mass density per cubic center for the agglomeration is 0.35 g/cm³ or about 0.35 g/cm³. In implementations, the maximum mass density for the agglomeration may be from about 0.34 g/cm³ to about 0.36 g/cm³, from about 0.33 g/cm³ to about 0.37 g/cm³, from about 0.32 g/cm³ to about 0.38 g/cm³, from about 0.31 g/cm³ to about 0.39 g/cm³, or from about 0.30 g/cm³ to about 0.40 g/cm³.

In various implementations, the composition may be manufactured using a dry-blending process to combine the resistant dextrin, the corosolic acid extract, and the chromium(III) picolinate. In such implementations, instead of forming the agglomerate, the dry-blending process may produce a dry mixture of these components. The dry mixture may then be combined with the flavor matrix and further processed, according to need.

After the agglomerate or dry mixture is formed, a flavored matrix, which may include one or more flavor agents and/or one or more flavor enhancing agents, may be added and dry-blended with the agglomerate (or added and dry-blended with the dry mixture). Accordingly, the method 5 may include dry blending 52 the agglomerate or the dry mixture with the flavored matrix to improve palatability of the composition. The flavored matrix may include a flavor agent, a flavor enhancing agent, or both. In various implementations, the flavor enhancing agent may include malic acid, citric acid, fumaric acid, tartaric acid, sucralose, or any combination thereof. While any of a number of systems and approaches may be used for dry blending, in particular implementations, a V-blender may be used with good results.

Accordingly, the disclosure provides a composition for metabolic health management manufactured by a method. The method includes wetly agglomerating or dry blending a plurality of ingredients to form an agglomerate or a dry mixture, and dry blending the agglomerate or the dry mixture with a flavored matrix to improve palatability of the composition. The plurality of ingredients includes resistant dextrin, a Lagerstroemia speciosa L. extract standardized to about 1% corosolic acid, and chromium(III) picolinate. The method may further include dividing the dietary supplement into a plurality of doses, such that each dose of the plurality of doses includes about 4500 mg to about 5000 mg of the resistant dextrin, about 0.48 mg to about 1.00 mg of the Lagerstroemia speciosa L. extract, and about 35 μg to about 200 μg of the chromium(III) picolinate. Alternatively, the method may further include dividing the dietary supplement into a plurality of doses, such that each dose of the plurality of doses includes about 4500 mg to about 5500 mg of the resistant dextrin, about 0.48 mg to about 1.00 mg of the Lagerstroemia speciosa L. extract, and about 35 μg to about 200 μg of the chromium(III) picolinate. In some embodiments, the method may further include dividing the dietary supplement into a plurality of doses, such that each dose of the plurality of doses includes about 5000 mg of the resistant dextrin, about 0.48 mg of the Lagerstroemia speciosa L. extract, and about 35 μg of the chromium(III) picolinate.

### Dose

A dose of the composition may be sized from about 4450 mg to about 4550 mg, from about 4460 mg to about 4540 mg, from about 4470 mg to about 4530 mg, from about 4480 mg to about 4520 mg, or from about 4490 to about 4510 mg. In certain implementations, the dose of the composition may be sized from about 4500.515 mg to about 5001.200 mg. The dose may be increased or decreased according to need and/or based on an evaluation of an individual's response to the dose as disclosed herein.

A dose of the composition may be sized from about 3000 mg to about 7000 mg, from about 3500 mg to about 6500 mg, about 400 mg to about 6000 mg, or from about 4500 to about 5500, from about 4800 to about 5200, from about 4900 to about 5100, from about 4950 mg to about 5050 mg, from about 4960 mg to about 5040 mg, from about 4970 mg to about 5030 mg, from about 4980 mg to about 5020 mg, or from about 4090 to about 5010 mg. In certain implementations, the dose of the composition may be sized from about 4500.515 mg to about 5001.200 mg. The dose may be increased or decreased according to need and/or based on an evaluation of an individual's response to the dose as disclosed herein.

### Treatment

In various implementations, a dose of a composition may be provided and/or administered to an individual as part of a treatment regimen. The treatment regimen may include lifestyle changes, such as changes in exercise and physical fitness, hours slept, hydration levels, and treatment for metabolic syndrome which includes obesity, T2DM, and cardiovascular disease. One or more doses of the composition may be administered to the individual once daily, twice daily, three times daily, or more times daily, according to need. The one or more doses of the composition may be administered daily, every other day, bi-weekly, bi-monthly, monthly, etc. according to a particular treatment regimen. The one or more doses of the composition may be administered before a meal, during a meal, and/or after a meal to attenuate post-prandial increases in plasma concentrations of glucose, triglycerides, and cholesterol, and/or to lower plasma concentrations of glucose, triglycerides, and cholesterol. The treatment may be intended for a mammal, such as a human being, a dog, a cat, and the like. In particular embodiments, the treatment may be intended for a human being and/or a group of human beings with the same or similar treatment needs.

### Dietary Supplements and Functional Foods or Beverages

In various implementations, one or more doses of the composition may be formulated and/or packaged into a dietary supplement and/or a functional food or beverage, such as a caffeine-free iced tea, a flavored drink mix, or a caffeine-free coffee, and may be in a powdered form or in a liquid form, or the same in a pill form, a tablet form, and the like. In certain implementations, the one or more doses of the composition may be formulated and/or packaged as a dietary supplement and/or a functional food (e.g., a snack bar) and/or a beverage (e.g., a smoothie or a shake). The product may be satisfactorily palatable to consumers, including pre-diabetic and those diagnosed with T2DM. The palatability of the product may be improved compared to previous efforts.

### EXAMPLES

Some aspects of the embodiments discussed above are disclosed in further detail in the following examples, which are not in any way intended to limit the scope of the present disclosure. Those in the art will appreciate that many other embodiments also fall within the scope of the compositions and methods of the present application, as is described herein above and in the claims.

### Example 1. Composition for Managing Metabolic Health

Materials used in preparing the compositions described herein may be made by known methods or are commercially available. It is also possible to make use of variants which are themselves known to those of ordinary skill in this art, but are not mentioned in greater detail. The skilled artisan given the literature and this disclosure is well equipped to prepare the formulations of the instant application.

Representative compositions are shown in Table 1 below, with the amounts for “broad”, “intermediate” and “preferred” ranges. The preferred amount indicates the optimal amount for each component.

TABLE 1
Representative Oral Compositions

| | Broad | Intermediate | Preferred |
|---|---|---|---|
| | Ranges | Ranges | Amounts |
| Component | (mg) | (mg) | (mg) |
| Resistant dextrin | 3500-6500 | 4500-5500 | 5000 |
| Lagerstroemia speciosa L. | 0.2-2.0 | 0.48-1.0 | 0.48 |
| (standardized to about 1% | | | |
| corosolic acid) | | | |
| chromium(III) picolinate | 0.015-0.250 | 0.035-0.200 | 0.035 |
| flavored matrix | as needed | as needed | as needed |

### Example 2

Subjects in need of managing metabolic health are administered the composition described herein in combination with a test meal. Initially, plasma concentrations of glucose, triglycerides, and cholesterol in the subject are measured before the ingestion of the meal. Periodically, after administration of the compositions disclosed herein, plasma concentrations of glucose, triglycerides, and cholesterol in the subject are measured at 0, 0.5, 1, 1.5, 2, 2.5, 3, 3.5, 4, 4.5, 5, 5.5, and/or 6 hours after the meal.

### EMBODIMENTS

The disclosure provides various embodiments, including compositions, methods for making compositions, and methods for using compositions, for improving an individual's health by attenuating post-prandial increases of, or lowering plasma concentrations of, glucose, triglycerides, and cholesterol in the individual. While these and other embodiments are explicitly disclosed, they are not intended to be the only embodiments contemplated herein. Other embodiments, including but not limited to implicitly disclosed embodiments as well as embodiments that may arise upon practicing disclosed embodiments are included within the scope of the present disclosure.

Embodiment 1: A composition for managing metabolic health, the composition comprising: resistant dextrin; an extract that comprises corosolic acid; chromium(III) picolinate; and a flavored matrix.

Embodiment 2: The composition of embodiment 1, wherein the extract that comprises corosolic acid is from Lagerstroemia speciosa L. and is standardized to about 1% corosolic acid.

Embodiment 3: The composition of embodiment 2, wherein a dose of the composition comprises: about 4500 mg to about 5500 mg of the resistant dextrin; about 0.48 mg to about 1.00 mg of the Lagerstroemia speciosa L. extract; and about 35 μg to about 200 μg of the chromium(III) picolinate.

Embodiment 4: The composition of embodiment 3, wherein the dose of the composition comprises: about 5000 mg of the resistant dextrin; about 0.48 mg of the Lagerstroemia speciosa L. extract; and about 35 μg of the chromium(III) picolinate.

Embodiment 5: The composition of embodiment 1, wherein the flavored matrix comprises a flavor agent, a flavor-enhancing agent, or both.

Embodiment 6: The composition of embodiment 5, wherein the flavor agent comprises malic acid, citric acid, fumaric acid, tartaric acid, sucralose, or any combination thereof.

Embodiment 7: The composition of embodiment 1, wherein the composition is manufactured by a method, the method comprising: wetly agglomerating or dry mixing the resistant dextrin, the extract that comprises the corosolic acid, and the chromium(III) picolinate to form an agglomerate or a dry mixture; and dry blending the agglomerate or the dry mixture with the flavored matrix.

Embodiment 8: A dietary supplement comprising the dose of the composition of embodiment 1.

Embodiment 9: A method for managing metabolic health, the method comprising: administering a dose of a composition to a mammal, the composition comprising: resistant dextrin; an extract that comprises corosolic acid; chromium(III) picolinate; and a flavored matrix; wherein administering the dose of the composition to the mammal attenuates a post-prandial increase of plasma concentrations of glucose, triglycerides, and cholesterol in the mammal; or lowers plasma concentrations of glucose, triglycerides, and cholesterol in the mammal.

Embodiment 10: The method of embodiment 9, wherein the extract that comprises corosolic acid is from Lagerstroemia speciosa L. and is standardized to about 1% corosolic acid.

Embodiment 11: The method of embodiment 10, wherein the dose of the composition comprises: about 4500 mg to about 5500 mg of the resistant dextrin; about 0.48 mg to about 1.00 mg of the Lagerstroemia speciosa L. extract; and about 35 μg to about 200 μg of the chromium(III) picolinate.

Embodiment 12: The method of embodiment 11, wherein the dose of the composition comprises: about 5000 mg of the resistant dextrin; about 0.48 mg of the Lagerstroemia speciosa L. extract; and about 35 μg of the chromium(III) picolinate.

Embodiment 13: The method of embodiment 9, further comprising: administering a meal to the mammal; measuring post-prandial plasma concentrations of glucose, triglycerides, and cholesterol in the mammal; and evaluating an attenuation of the post-prandial increase of plasma concentrations of glucose, triglycerides, and cholesterol in the mammal.

Embodiment 14: The method of embodiment 13, further comprising: adjusting the dose of the composition based on an evaluation of the attenuation of the post-prandial increase of plasma concentrations of glucose, triglycerides, and cholesterol in the mammal.

Embodiment 15: A method for manufacturing a composition for metabolic health management, the method comprising: wetly agglomerating or dry mixing resistant dextrin, an extract that comprises corosolic acid, and chromium(III) picolinate to form an agglomerate or a dry mixture.

Embodiment 16: The method of embodiment 15, wherein the extract that comprises corosolic acid is from Lagerstroemia speciosa L. and is standardized to about 1% corosolic acid.

Embodiment 17: The method of embodiment 16, further comprising: dividing the composition into a plurality of doses, wherein each dose of the plurality of doses comprises: about 4500 mg to about 5500 mg of the resistant dextrin; about 0.48 mg to about 1.00 mg of the Lagerstroemia speciosa L. extract; and about 35 μg to about 200 μg of the chromium(III) picolinate.

Embodiment 18: The method of embodiment 17, wherein each dose of the plurality of doses comprises: about 5000 mg of the resistant dextrin; about 0.48 mg of the Lagerstroemia speciosa L. extract; and about 35 μg of the chromium(III) picolinate.

Embodiment 19: The method of embodiment 15, further comprising: dry blending the agglomerate or the dry mixture with a flavored matrix to improve palatability of the composition, wherein the flavored matrix comprises a flavor agent, a flavor-enhancing agent, or both.

Embodiment 20: The method of embodiment 19, wherein the flavor agent comprises malic acid, citric acid, fumaric acid, tartaric acid, sucralose, or any combination thereof.

Embodiment 21: A composition for managing metabolic health, the composition consisting essentially of: resistant dextrin; a Lagerstroemia speciosa L. extract standardized to about 1% corosolic acid; chromium(III) picolinate; and a flavored matrix; wherein an administration of a dose of the composition to an individual attenuates a post-prandial increase of plasma concentrations of glucose, triglycerides, and cholesterol in the individual; or lowers plasma concentrations of glucose, triglycerides, and cholesterol in the individual.

Embodiment 22: The composition of embodiment 21, wherein the dose of the composition consists essentially of: about 4500 mg to about 5500 mg of the resistant dextrin; about 0.48 mg to about 1.00 mg of the Lagerstroemia speciosa L. extract; about 35 μg to about 200 μg of the chromium(III) picolinate; and the flavored matrix.

Embodiment 23: The composition of embodiment 22, wherein the dose of the composition consists essentially of: about 5000 mg of the resistant dextrin; about 0.48 mg of the Lagerstroemia speciosa L. extract; about 35 μg of the chromium(III) picolinate; and the flavored matrix.

Embodiment 24: A dietary supplement consisting essentially of the dose of the composition of embodiment 21.

Embodiment 25: A composition for managing metabolic health, the composition consisting of: resistant dextrin; a Lagerstroemia speciosa L. extract standardized to about 1% corosolic acid; chromium(III) picolinate; and a flavored matrix to improve palatability of the composition; wherein an administration of a dose of the composition to an individual attenuates a post-prandial increase of plasma concentrations of glucose, triglycerides, and cholesterol in the individual; or lowers plasma concentrations of glucose, triglycerides, and cholesterol in the individual.

Embodiment 26: The composition of embodiment 25, wherein the dose of the composition consists of: about 4500 mg to about 5500 mg of the resistant dextrin; about 0.48 mg to about 1.00 mg of the Lagerstroemia speciosa L. extract; about 35 μg to about 200 μg of the chromium(III) picolinate; and the flavored matrix.

Embodiment 27: The composition of embodiment 26, wherein the dose of the composition consists of: about 5000 mg of the resistant dextrin; about 0.48 mg of the Lagerstroemia speciosa L. extract; about 35 μg of the chromium(III) picolinate; and the flavored matrix.

Embodiment 28: A dietary supplement consisting of the dose of the composition of embodiment 25.

Embodiment 29: A dietary supplement for metabolic health management manufactured by a method, the method comprising: wetly agglomerating or dry mixing a plurality of ingredients to form an agglomerate or a dry mixture, the plurality of ingredients comprising: resistant dextrin; a Lagerstroemia speciosa L. extract standardized to about 1% corosolic acid; and chromium(III) picolinate; dry blending the agglomerate or the dry mixture with a flavored matrix to improve palatability of the dietary supplement; and dividing the dietary supplement into a plurality of doses, wherein each dose of the plurality of doses comprises: about 4500 mg to about 5500 mg of the resistant dextrin; about 0.48 mg to about 1.00 mg of the Lagerstroemia speciosa L. extract; and about 35 μg to about 200 μg of the chromium(III) picolinate.

Embodiment 30: The dietary supplement of embodiment 29, wherein each dose of the plurality of doses comprises: about 5000 mg of the resistant dextrin; about 0.48 mg of the Lagerstroemia speciosa L. extract; and about 35 μg of the chromium(III) picolinate.

Embodiment 31: A dietary supplement for metabolic health management manufactured by a method, the method consisting essentially of: wetly agglomerating or dry mixing a plurality of ingredients to form an agglomerate or a dry mixture, the plurality of ingredients consisting essentially of: resistant dextrin; a Lagerstroemia speciosa L. extract standardized to about 1% corosolic acid; and chromium(III) picolinate; dry blending the agglomerate or the dry mixture with a flavored matrix to improve palatability of the dietary supplement; and dividing the dietary supplement into a plurality of doses, wherein each dose of the plurality of doses consists essentially of: about 4500 mg to about 5500 mg of the resistant dextrin; about 0.48 mg to about 1.00 mg of the Lagerstroemia speciosa L. extract; about 35 μg to about 200 μg of the chromium(III) picolinate; and the flavored matrix.

Embodiment 32: The composition of embodiment 31, wherein the dose of the composition consists essentially of: about 5000 mg of the resistant dextrin; about 0.48 mg of the Lagerstroemia speciosa L. extract; about 35 μg of the chromium(III) picolinate; and the flavored matrix.

Embodiment 33: A dietary supplement for metabolic health management manufactured by a method, the method consisting of: wetly agglomerating or dry mixing a plurality of ingredients to form an agglomerate or a dry mixture, the plurality of ingredients consisting of: resistant dextrin; a Lagerstroemia speciosa L. extract standardized to about 1% corosolic acid; and chromium(III) picolinate; dry blending the agglomerate or the dry mixture with a flavored matrix to improve palatability of the dietary supplement; and dividing the dietary supplement into a plurality of doses, wherein each dose of the plurality of doses consists of: about 4500 mg to about 5500 mg of the resistant dextrin; about 0.48 mg to about 1.00 mg of the Lagerstroemia speciosa L. extract; about 35 μg to about 200 μg of the chromium(III) picolinate; and the flavored matrix.

Embodiment 34: The composition of embodiment 31, wherein the dose of the composition consists of: about 5000 mg of the resistant dextrin; about 0.48 mg of the Lagerstroemia speciosa L. extract; about 35 μg of the chromium(III) picolinate; and the flavored matrix.

The foregoing descriptions of specific implementations have been presented for purposes of illustration and description. They are not intended to be exhaustive or to limit the disclosure to the precise forms disclosed, and modifications and variations are possible in view of the above teaching. The exemplary implementations were chosen and described to best explain the principles of the disclosure and its practical application, to thereby enable others skilled in the art to best utilize the disclosure and its implementations with modifications as suited to the use contemplated.

It is therefore submitted that the disclosure has been shown and described in the most practical and exemplary implementations. It should be recognized that departures may be made which fall within the scope of the disclosure. With respect to the description provided herein, it is submitted that the optimal features of the disclosure include variations in size, materials, shape, form, function, manner of operation, assembly, and use. All structures, functions, and relationships equivalent or essentially equivalent to those disclosed are intended to be encompassed by the disclosure.

What is claimed is:

1. A composition for managing metabolic health, the composition comprising:
resistant dextrin;
an extract that comprises corosolic acid;
chromium(III) picolinate; and
a flavored matrix.

2. The composition of claim 1, wherein the extract that comprises corosolic acid is from Lagerstroemia speciosa L. and is standardized to about 1% corosolic acid.

3. The composition of claim 2, wherein a dose of the composition comprises:
about 4500 mg to about 5500 mg of the resistant dextrin;
about 0.48 mg to about 1.00 mg of the Lagerstroemia speciosa L. extract; and
about 35 μg to about 200 μg of the chromium(III) picolinate.

4. The composition of claim 3, wherein the dose of the composition comprises:
about 5000 mg of the resistant dextrin;
about 0.48 mg of the Lagerstroemia speciosa L. extract; and
about 35 μg of the chromium(III) picolinate.

5. The composition of claim 1, wherein the flavored matrix comprises a flavor agent, a flavor-enhancing agent, or both.

6. The composition of claim 5, wherein the flavor agent comprises malic acid, citric acid, fumaric acid, tartaric acid, sucralose, or any combination thereof.

7. The composition of claim 1, wherein the composition is manufactured by a method, the method comprising:
wetly agglomerating or dry mixing the resistant dextrin, the extract that comprises the corosolic acid, and the chromium(III) picolinate to form an agglomerate or a dry mixture; and
dry blending the agglomerate or the dry mixture with the flavored matrix.

8. A dietary supplement comprising the dose of the composition of claim 1.

9. A method for managing metabolic health, the method comprising:
administering a dose of a composition to a mammal, the composition comprising:
resistant dextrin;
an extract that comprises corosolic acid;
chromium(III) picolinate; and
a flavored matrix;
wherein administering the dose of the composition to the mammal attenuates a post-prandial increase of plasma concentrations of glucose, triglycerides, and cholesterol in the mammal; or
lowers plasma concentrations of glucose, triglycerides, and cholesterol in the mammal.

10. The method of claim 9, wherein the extract that comprises corosolic acid is from Lagerstroemia speciosa L. and is standardized to about 1% corosolic acid.

11. The method of claim 10, wherein the dose of the composition comprises:
about 4500 mg to about 5500 mg of the resistant dextrin;
about 0.48 mg to about 1.00 mg of the Lagerstroemia speciosa L. extract; and
about 35 μg to about 200 μg of the chromium(III) picolinate.

12. The method of claim 11, wherein the dose of the composition comprises:
about 5000 mg of the resistant dextrin;
about 0.48 mg of the Lagerstroemia speciosa L. extract; and
about 35 μg of the chromium(III) picolinate.

13. The method of claim 9, further comprising:
administering a meal to the mammal;
measuring post-prandial plasma concentrations of glucose, triglycerides, and cholesterol in the mammal; and
evaluating an attenuation of the post-prandial increase of plasma concentrations of glucose, triglycerides, and cholesterol in the mammal.

14. The method of claim 13, further comprising:
adjusting the dose of the composition based on an evaluation of the attenuation of the post-prandial increase of plasma concentrations of glucose, triglycerides, and cholesterol in the mammal.

15. A method for manufacturing a composition for metabolic health management, the method comprising:
wetly agglomerating or dry mixing resistant dextrin, an extract that comprises corosolic acid, and chromium(III) picolinate to form an agglomerate or a dry mixture.

16. The method of claim 15, wherein the extract that comprises corosolic acid is from Lagerstroemia speciosa L. and is standardized to about 1% corosolic acid.

17. The method of claim 16, further comprising:
dividing the composition into a plurality of doses, wherein each dose of the plurality of doses comprises:
about 4500 mg to about 5500 mg of the resistant dextrin;
about 0.48 mg to about 1.00 mg of the Lagerstroemia speciosa L. extract; and
about 35 μg to about 200 μg of the chromium(III) picolinate.

18. The method of claim 17, wherein each dose of the plurality of doses comprises:
about 5000 mg of the resistant dextrin;
about 0.48 mg of the Lagerstroemia speciosa L. extract; and
about 35 μg of the chromium(III) picolinate.

19. The method of claim 15, further comprising:
dry blending the agglomerate or the dry mixture with a flavored matrix to improve palatability of the composition, wherein the flavored matrix comprises a flavor agent, a flavor-enhancing agent, or both.

20. The method of claim 19, wherein the flavor agent comprises malic acid, citric acid, fumaric acid, tartaric acid, sucralose, or any combination thereof.

21. A composition for managing metabolic health, the composition consisting essentially of:
resistant dextrin;
a Lagerstroemia speciosa L. extract standardized to about 1% corosolic acid;
chromium(III) picolinate; and
a flavored matrix;
wherein an administration of a dose of the composition to an individual attenuates a post-prandial increase of plasma concentrations of glucose, triglycerides, and cholesterol in the individual; or
lowers plasma concentrations of glucose, triglycerides, and cholesterol in the individual.

22. The composition of claim 21, wherein the dose of the composition consists essentially of:
about 4500 mg to about 5500 mg of the resistant dextrin;
about 0.48 mg to about 1.00 mg of the Lagerstroemia speciosa L. extract;
about 35 μg to about 200 μg of the chromium(III) picolinate; and
the flavored matrix.

23. The composition of claim 22, wherein the dose of the composition consists essentially of:
about 5000 mg of the resistant dextrin;
about 0.48 mg of the Lagerstroemia speciosa L. extract;
about 35 μg of the chromium(III) picolinate; and
the flavored matrix.

24. A dietary supplement consisting essentially of the dose of the composition of claim 21.

25. A composition for managing metabolic health, the composition consisting of:
resistant dextrin;
a Lagerstroemia speciosa L. extract standardized to about 1% corosolic acid;
chromium(III) picolinate; and
a flavored matrix to improve palatability of the composition;
wherein an administration of a dose of the composition to an individual attenuates a post-prandial increase of plasma concentrations of glucose, triglycerides, and cholesterol in the individual; or
lowers plasma concentrations of glucose, triglycerides, and cholesterol in the individual.

26. The composition of claim 25, wherein the dose of the composition consists of:
about 4500 mg to about 5500 mg of the resistant dextrin;
about 0.48 mg to about 1.00 mg of the Lagerstroemia speciosa L. extract;
about 35 μg to about 200 μg of the chromium(III) picolinate; and
the flavored matrix.

27. The composition of claim 26, wherein the dose of the composition consists of:
about 5000 mg of the resistant dextrin;
about 0.48 mg of the Lagerstroemia speciosa L. extract;
about 35 μg of the chromium(III) picolinate; and
the flavored matrix.

28. A dietary supplement consisting of the dose of the composition of claim 25.

29. A dietary supplement for metabolic health management manufactured by a method, the method comprising:
wetly agglomerating or dry mixing a plurality of ingredients to form an agglomerate or a dry mixture, the plurality of ingredients comprising:
resistant dextrin;
a Lagerstroemia speciosa L. extract standardized to about 1% corosolic acid; and
chromium(III) picolinate;
dry blending the agglomerate or the dry mixture with a flavored matrix to improve palatability of the dietary supplement; and
dividing the dietary supplement into a plurality of doses, wherein each dose of the plurality of doses comprises:
about 4500 mg to about 5500 mg of the resistant dextrin;
about 0.48 mg to about 1.00 mg of the Lagerstroemia speciosa L. extract; and
about 35 μg to about 200 μg of the chromium(III) picolinate.

30. The dietary supplement of claim 29, wherein each dose of the plurality of doses comprises:
about 5000 mg of the resistant dextrin;
about 0.48 mg of the Lagerstroemia speciosa L. extract; and
about 35 μg of the chromium(III) picolinate.

31. A dietary supplement for metabolic health management manufactured by a method, the method consisting essentially of:
wetly agglomerating or dry mixing a plurality of ingredients to form an agglomerate or a dry mixture, the plurality of ingredients consisting essentially of:
resistant dextrin;
a Lagerstroemia speciosa L. extract standardized to about 1% corosolic acid; and
chromium(III) picolinate;
dry blending the agglomerate or the dry mixture with a flavored matrix to improve palatability of the dietary supplement; and
dividing the dietary supplement into a plurality of doses, wherein each dose of the plurality of doses consists essentially of:
about 4500 mg to about 5500 mg of the resistant dextrin;
about 0.48 mg to about 1.00 mg of the Lagerstroemia speciosa L. extract;
about 35 μg to about 200 μg of the chromium(III) picolinate; and
the flavored matrix.

32. The composition of claim 31, wherein the dose of the composition consists essentially of:
about 5000 mg of the resistant dextrin;
about 0.48 mg of the Lagerstroemia speciosa L. extract;
about 35 μg of the chromium(III) picolinate; and
the flavored matrix.

33. A dietary supplement for metabolic health management manufactured by a method, the method consisting of:
wetly agglomerating or dry mixing a plurality of ingredients to form an agglomerate or a dry mixture, the plurality of ingredients consisting of:
resistant dextrin;
a Lagerstroemia speciosa L. extract standardized to about 1% corosolic acid; and
chromium(III) picolinate;
dry blending the agglomerate or the dry mixture with a flavored matrix to improve palatability of the dietary supplement; and
dividing the dietary supplement into a plurality of doses, wherein each dose of the plurality of doses consists of:
about 4500 mg to about 5500 mg of the resistant dextrin;
about 0.48 mg to about 1.00 mg of the Lagerstroemia speciosa L. extract;
about 35 μg to about 200 μg of the chromium(III) picolinate; and
the flavored matrix.

34. The composition of claim 31, wherein the dose of the composition consists of:
about 5000 mg of the resistant dextrin;
about 0.48 mg of the Lagerstroemia speciosa L. extract;
about 35 μg of the chromium(III) picolinate; and
the flavored matrix.
